(19) United States
(12) Patent Application Publication
(10) Pub. No.: US 2014/0236156 A1
(43) Pub. Date: Aug. 21, 2014

(54) SURGICAL TOOL FOR REAMING THE DIAPHYSEAL CANAL OF LONG BONES

(71) Applicants: Yvan Arlettaz, Monthey (CH); Christian Bonjour, Begnins (CH)

(72) Inventors: Yvan Arlettaz, Monthey (CH); Christian Bonjour, Begnins (CH)

(73) Assignee: CHIRMAT SÀRL, Monthey (CH)

(21) Appl. No.: 14/345,087

(22) PCT Filed: Sep. 4, 2012
(86) PCT No.: PCT/EP2012/067240

§ 371 Date: Apr. 28, 2014

(30) Foreign Application Priority Data
Sep. 16, 2011 (CH) 1536/2011

(51) Int. Cl. A61B 17/16

(52) U.S. Cl. CPC A61B 17/164; A61B 17/1615; A61B 2017/00526
USPC 606/80; 76/108.1

(57) ABSTRACT

The invention relates to a surgical tool (10) for reaming the diaphyseal canal of long bones, in particular of the femur, humerus, or tibia, in order to insert an intramedullary rod. The modular reaming head includes: a central core (60) and a peripheral bushing (24) that is coaxial to said central core (60) and connected via a plurality of vanes (41a-41e) connecting a front surface (12) of the tool (10) to a rear surface (11) of the tool (10), and teeth (26a-26e) on the outer surface of said peripheral bushing (24). The openings allow material to pass toward the rear when the tool advances within the diaphyseal canal, minimizing pressure and friction, resulting in a positive effect on the rate of clinical complications. The tool is preferably made using the metal injection molding (MIM) technique in order to reduce production costs and enable the one-time use thereof. The reaming head of the invention can be perfectly combined with composite or metal drive shafts.

### REFERENCE DATA

This patent application is an entry into national US phase of international patent application PCT/EP2012/067240 of Sep. 4, 2012 claiming priority of Swiss patent application CH153672011 of Sep. 16, 2011, which is hereby incorporated in its entirety by reference.

### TECHNICAL FIELD

The present invention relates to a reaming head for enlarging the medullar canal for the treatment of fractures of long bones.

### STATE OF THE ART

Centro-medullar nailing is one of the most widely used therapeutic techniques in the treatment of fractures and in the reconstruction of long bones, notably in the case of factures of the femur body, but this technique can also be applied to other long bones such as the humerus or the tibia. The centro-medullar reaming technique consists in enlarging the diaphyseal canal by means of a reaming head or a series of reaming heads of increasing diameter, mounted on a flexible drive shaft. The reaming operation occurs prior to the insertion of a metallic nail or pin into the diaphyseal canal in order to set and stabilize the fracture and simultaneously relieve the compression on the bone.

Centro-medullar reaming, by achieving a calibration to the desired diameter of the diaphyseal canal, improves the adaptation and adhesion of the nail to the wall of the medullary canal and considerably reduces the risk of the nail becoming jammed or the body of the long bone bursting during insertion of the implant.

In addition to the above-mentioned mechanical advantages, this technique also affords considerable advantages biologically and clinically: it can notably transport bone particles on the site of The fracture, with an effect equivalent to a self-graft, which contributes to the healing process.

In order to ensure that the reaming is perfectly concentric relative to the medullary canal, a guiding rod (also known by the name of Kirschner wire) is previously inserted until the metaphysis that is distal relative to the fracture. The reaming head and its drive shaft have an axial hole for sliding concentrically on the guiding rod. The guiding rods are preferably equipped with an olive or a protuberance at the distal extremity in order to recover, if necessary, a reaming head that has become blocked or damaged.

This technique is also advisable due to its undeniable clinical advantages. It also has some disadvantages linked notably to the pressure and heating that can occur inside the medullar canal during the reaming and which could cause fat embolisms. An appropriate surgical technique with moderate cutting and advancing speeds and which limits the removed thickness to 0.2 to 0.5 mm (at the radius) per run makes it possible to reduce these risks, without however eliminating them completely.

Specific tools for reaming the medullary canal, for example reamers described by documents EP0648477, EP2335617, EP1987785, are known. Document US2004236339 describes a guiding device for centro-medullar reaming, whilst US2006064164 discloses orthopedic drills used prior to the implant placement in the case of a femoral neck fracture. Yet these devices are costly and must be re-used. They are, however, difficult to clean and sterilize, particularly in a hospital environment. Their use is challenging due to the overpressures they cause in the bone during operation and the friction-induced overheating they generate, particularly if their cutting edges are not perfectly sharp.

### BRIEF SUMMARY OF THE INVENTION

One aim of the present invention is to propose a reaming head free from the limitations of the known tools, and notably a reaming head that enables the overpressure and overheating effects to be minimized during the enlargement of the medullary canal.

Another aim of the invention is to propose a reaming head that is more economical than the known devices, making its single use possible.

According to the invention, these aims are achieved notably by means of the object of the attached claims, and notably by a surgical tool for reaming the diaphyseal canal of long bones comprising: a central core and a peripheral bushing that is coaxial to said central core and connected via a plurality of vanes defining a plurality of axial openings connecting a front surface of the tool to a rear surface of the tool, and teeth on the outer surface of said peripheral bushing.

### BRIEF DESCRIPTION OF THE FIGURES

Examples of embodiments of the invention are indicated in the description illustrated by the attached figures, wherein:

FIGS. 1a and 1d illustrate a reaming head according to one aspect of the invention, seen from the front side resp. from the rear side;

FIGS. 1b and 1c illustrate the reaming head of FIGS. 1a and 1d seen from the side and from the front;

FIG. 2 illustrates the angles characteristic of the reaming head of FIGS. 1a-1d;

FIG. 3 illustrates a possible connection system between a reamer according to the invention and a drive shaft.

### EXAMPLE(S) OF EMBODIMENTS OF THE INVENTION

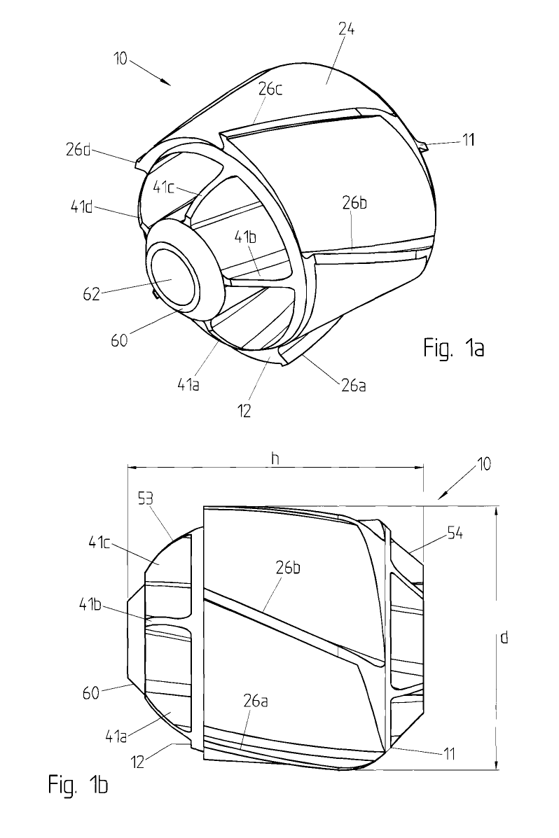

With reference to FIGS. 1a to 1d, a reamer according to one aspect of the invention comprises a cylindrical truncated-cone shaped bushing 24 bearing helical teeth 26a-26e on its outer surface. The radius r of the reaming head 10 is chosen according to the dimensions of the bone to be treated and to the canal diameter that is to be achieved. The reaming heads are preferably made in series with progressively increasing diameters, for example with a radius progression of 0.2-0.5 mm between two successive elements. This enables the surgeon to perform the reaming in several runs, removing a small quantity of cortical bone each time. In the case of a fracture of the femoral body, it is generally recommended to bring the medullary canal to a final diameter of between 11 and 14 mm before inserting the nail, but several factors can influence these dimensions, e.g. the size of the patient, the nature of the fracture and the type of nail among others.

The height of the reaming head measured along the rotation axis is chosen relative to its lateral dimensions, being preferably comprised between 5 and 20 mm depending on the diameter of the reaming head.

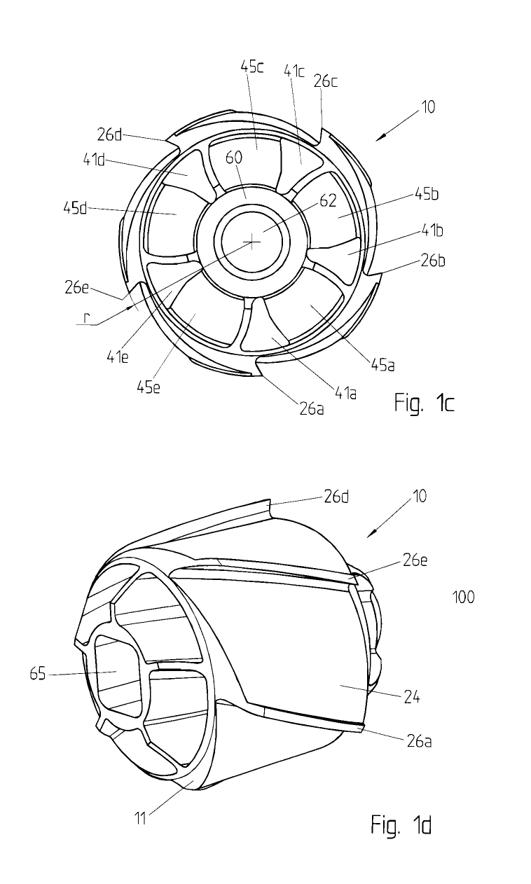

FIG. 2 shows the profile of one of the helical teeth 26a-e. The teeth preferably have a substantial cutting angle α to reduce the cutting force, and also a pronounced clearing angle δ so as to minimize the friction of the tooth's rear part on the machined part. The wedge angle β, determined by the relation α+β+δ=90°, is consequently moderate, but not to the point of weakening the tooth. Tests have lead to the conclusion that angles comprised between 15° and 40°, typically between 20° and 30° for α and between 3° and 20°, typically between 7° and 15°, for δ, afford optimum performance. The helical angle of the teeth is preferably comprised between 15° and 35°. Surgical reaming heads are generally designed to turn in a clockwise direction, and the helical angle is consequently to the right, so as to extract the cut material towards the rear of the tool.

The figures refer to the case of a tool with five teeth, but it will be clearly appreciated that this is not a limiting characteristic of the invention. The reaming head can bear a different number of teeth, for example two, three or four, even six or more.

Reverting now to FIG. 1, the reaming head has a core 60 that is coaxial with the bushing 24 with a central hole 62 enabling the Kirschner wire to pass. In this embodiment, the axial hole 62 of the front side has a round section (FIG. 1a) whose diameter corresponds to that of the Kirschner wire, whilst on the rear side, the reaming head has for example an axial hole 65 with a polygonal section (FIG. 1d) to enable the motor torque to be transmitted from a drive shaft, as will be seen further below. Other forms would also be possible within the frame of the invention.

The core 60 and the bushing 24 are integrally united in a rigid manner by radial vanes 41a-41e that leave open a large part of the surface between the core 60 and the bushing 24. In the drawings, the reamer has five vanes and four axial openings 45a-45e, but this is riot a necessary characteristic of the invention, the vanes and openings can be in any number whatsoever.

The effect of the axial openings 45a-e is to allow the bone marrow to pass from the front to the back of the tool when the latter advances in the diaphyseal canal, thus alleviating the overpressure in the canal and reducing the risk of fat embolisms or bursting.

It is thus possible to assimilate the diaphyseal canal to a cylinder with a constant diameter having a porous distal extremity filled with a high-viscosity liquid, incompressible in regard to any practical effects, and the reaming head to a disc that is pushed inside from the proximal extremity. The flow of internal material is determined by the equations of continuity and of the conservation of mass. There are three interesting cases:
If the disc is full and the play between the disc and the wall is very small, the pressure will strongly increase in the cylinder; part of the viscous liquid will exit through the porous extremity and another part through the play between the head and the wall: the overpressure causes part of the fluid to pass into the blood circulation and this increases thus the risk of fat embolism.
If the disc is full and there is a considerable play between the disc and the wall, the pressure will increase but will not be sufficient to cause the viscous liquid to percolate through the porous extremity, the piston's displacement will be difficult and will cause overheating through friction of the liquid (speed ratio to surface ratio).
If however the disc is perforated and has axial openings enabling the front side to communicate with the rear side, and the exit towards its rear side is not blocked, the pressure will nearly not increase, there will be no percolation occurring at the porous extremity nor will the temperature rise since the majority of the viscous liquid will escape through the center of the disc.

It can thus be seen how the structure of the inventive reaming head enables bone marrow to pass through the openings 45a-45e, thus ensuring that it moves more easily and that the pressure inside the diaphyseal canal is reduced. In this manner, the inventive reaming head facilitates the surgical operation and reduces the probability of embolism or other unfavorable outcomes.

According to a preferred embodiment of the invention, the vanes 41a-e are not contained within axial planes but are curved or inclined so as to enhance the extraction of the marrow towards the rear side through the rotation of the tool without compressing it, in the manner of an auger screw. The inclined or curved arrangement also increases the tool's rigidity relative to a structure with plane vanes.

In a preferred embodiment, the front side of the reaming head is not plane but includes a convergent section 53, for example with a domed or truncated cone profile, which makes it easier to center the tool in the diaphyseal canal. The rear side preferably also comprises a convergent section 54, for example with a domed or truncated cone profile, whose aim is to reduce the tool's friction.

The inventive tool is preferably made of surgical steel or of any other suitable material and is preferably intended for single use (but not exclusively), so that the surgeon has for each reaming operation a perfectly sharpened tool, which considerably reduces the cutting efforts, friction and heating. It is preferably made using the MIM (Metal Injection Molding) technique in order to reduce its cost price.

The central core 60 preferably has a small diameter so as to maximize the section of the openings. The core 60 will preferably have a diameter of less than 7.0 mm, even more preferably equal to or less than 5.0 mm; the core's diameter stands in relation to the diameter of the reaming head. The drive shaft and the support designed to fixedly unite the reaming head to the shaft preferably also have limited diameters so as to not impede the flow of the marrow. Supports will be used that have a diameter of less than 9.0 mm preferably less than 8.0 mm; the diameter of the drive shaft stands in relation to the long bone that is reamed. The present invention can be used with several shapes of drive shaft and supports.

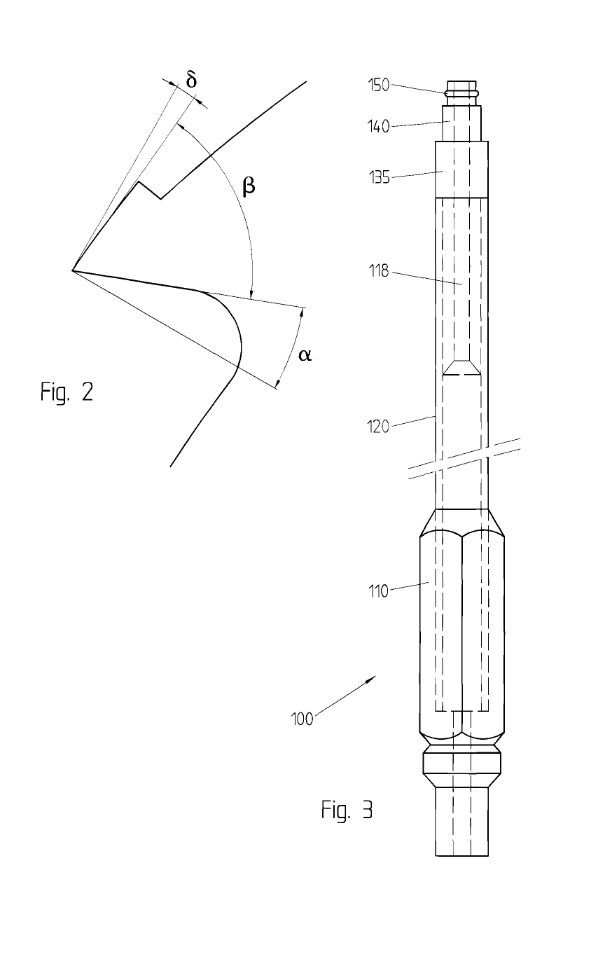

The inventive reaming head, preferably for single use, would preferably be coupled with a single use drive shaft made of composite material, such as for example the devices described in International patent application WO2011051260 in the name of the applicant and represented in FIG. 3.

The drive shaft 100 is completely traversed axially by an axial channel 118 designed to allow the Kirschner wire to go through. At one extremity, it offers an interface element 110 for coupling with the chuck of a motor, for example a hexagonal prismatic element, or any other suitable interface element. The trunk 120 of the drive shaft is preferably a flexible rod made of composite material comprising carbon and/or glass fibers encapsulated in a resin matrix containing an elastomer. This type of embodiment makes it possible to propose drive shafts with optimum elasticity and resistance characteristics at a very advantageous cost. The inventive reaming head, however, can be used in combination with any type of drive shaft: it would for example be possible to couple it with classical metallic transmission shafts or with composite transmission shafts having different characteristics, while remaining within the scope of the invention.

The connection between the shaft 100 and the reaming heads is achieved, in this embodiment, by a connection element 135 comprising a tenon 140 having a polygonal section that marries the shape of the axial hole 65 of the rear side of the reaming head. The elastic ring 150 that engages in a corresponding groove (not visible in the figures) of the reaming head ensures stability in the axial direction.

This arrangement enables the reaming heads to be exchanged easily and the motor torque to be transmitted in an excellent fashion whilst remaining very compact. The invention is however not limited to the coupling system described here above and represented in the figures, but also includes tenons of any shape, for example dovetail, wedges/cotter pins, screw assemblies or any other suitable coupling system.

It is thus possible to propose a complete range of reaming heads and a transmission shaft in the form of a single-use kit, thus eliminating all cleaning and sterilizing problems of the known tools and always providing sharpened and perfectly performing tools.

### REFERENCE NUMBERS USED IN THE FIGURES

α cutting angle
β wedge angle
δ clearing angle
10 reaming head
11 rear side
12 front side
24 bushing
26a-e teeth
41a-e vanes
45a-e axial openings
53 front side convergent section
54 rear side convergent section
60 core
62 front side axial hole
65 rear side axial hole
100 drive shaft
110 motor interface
118 axial channel
120 flexible rod
135 connecting element
140 tenon with polygonal section
150 elastic ring

1. A surgical tool for reaming the diaphyseal canal of long bones, comprising: a central core et and a peripheral bushing that is coaxial to said central core and connected via a plurality of vanes defining a plurality of axial openings connecting a front surface of the tool to a rear surface of the tool, and teeth on the outer surface of said peripheral bushing.

2. The surgical tool of claim 1, wherein said vanes have curved or inclined surfaces.

3. The surgical tool of claim 1, wherein said core comprises an axial hole.

4. The surgical tool of claim 1, wherein said teeth are arranged in a helical fashion.

5. The surgical tool of claim 1, wherein said bushing has an essentially cylindrical or truncated-cone shape.

6. The surgical tool of claim 1 in combination with a transmission shaft having a flexible rod of composite with carbon and/or glass fibers encapsulated in a polymeric matrix comprising an elastomer.

7. Use of the metal injection molding (MIM) technique for making the surgical tool for reaming the diaphyseal canal of long bones, said surgical tool comprising: a central core et and a peripheral bushing that is coaxial to said central core and connected via a plurality of vanes defining a plurality of axial openings connecting a front surface of the tool to a rear surface of the tool, and teeth on the outer surface of said peripheral bushing.
